(19) United States
(12) Patent Application Publication
(10) Pub. No.: US 2010/0297671 A1
(43) Pub. Date: Nov. 25, 2010

(54) Method and Device for the Quantitative Determination of Analytes in Liquid Samples

(76) Inventors: Jens Tschmelak, Erlangen (DE); Günther Proll, Altbach (DE); Nina Käppel, Rottenburg (DE); Günther Gauglitz, Tubingen (DE)

Correspondence Address: KF ROSS PC, 5683 RIVERDALE AVENUE, SUITE 203 BOX 900, BRONX, NY, 10471-0900, US

(21) Appl. No.: 11/921,971

(22) PCT Filed: May 26, 2006
(86) PCT No.: PCT/EP2006/005078

§ 371 Date: Jun. 30, 2010

(30) Foreign Application Priority Data
Jun. 10, 2005 (DE) 102005026839.0

(51) Int. Cl. G01N 33/53

(52) U.S. Cl. 435/7.21; 436/501

(57) ABSTRACT

The invention relates to a method and a device for the highly sensitive parallel detection and quantitative determination of analytes in liquid samples. According to said method, total internal reflection fluorescence (TIRF) is used in combination with a binding inhibition test on a specially coated support. The inventive method makes it possible to quickly analyze different types of liquids, such as drinking water, fruit juices, milk, serum, blood plasma, urine, etc., while allowing samples to be analyzed simultaneously regarding several different analytes, including hormones, antibiotics, pesticides, pharmaceuticals, drugs, and other molecules or molecular complexes, for example.

The present invention is directed to a method and a device for highly sensitive parallel detection of analytes in liquid samples.

Until now, analysis of liquid samples have been conducted predominantly by cost-intensive chromatographic methods with complex specimen preparation (R. J. Kavlock et al. (1996) Environ Health Perspect 104: 715-740; M. Petrovic et al. (2002) J Chromatogr A 974: 23-51; R. Heinrich-Ramm et al. (2004) Anal Bioanal Chem 380: 59-67; G. S. Pope, J. K. Swinburne (1980) J Dairy Res 47: 427-449; T. Mottram et al. (2002) Comp Clin Path 1 1:50-58).

Disadvantages of the common methods can be seen above all in the cost-intensive specimen preparation which is caused by the need for concentrating the liquid and a possible derivation of the analytes. In addition, problems arise from different applications because conventional methods depend highly on the matrix. Therefore different approaches are required for the measurement of different categories of substances.

This is why the problem of the present invention is to provide a low-cost method and a device for the fast, highly sensitive and parallel detection of several different analytes.

This problem is solved by the method with the characteristics of claim 1. Preferred embodiments of this method are shown in the dependent claims 2 to 26. The device, according to the present invention, is described in claim 27. Preferential uses contains claim 28. The wording of all claims is done hereby in reference to the content of this specification.

According to the present invention, the total internal reflection-fluorescence (TIRF) is established on a special coated support in combination with a binding inhibition test.

Hereunto, layers are applied first on the surface of a support. A light conductive medium is used as a support material. Especially glass or plastic are appropriate materials for this purpose. In a particular preferred embodiment the support shows two parallel surfaces.

Preferably, first of all, a layer of polymers is applied according to the method as described in DE 19816604 A1. Then, according to the present invention, a layer is applied that comprises substances with molecular structures which correspond to or resemble the analytes to be analyzed. Molecular structures of the applied substances have thereby appropriate characteristics to be detected sufficiently and specifically by means of structures of recognition of the ligands added later in the binding test of the assay. Thereby, the applied substances can correspond to the assayed analyte or to a derivative of the analyte whereas these substances show appropriate functional groups to be bond to the polymer layer in a covalent or non-covalent manner. If only one analyte shall be detected, the corresponding molecular structures are applied two-dimensionally. When there are several analytes to be assayed, the application of different molecular structures is performed in a spatially separated manner, e.g. in the form of spots.

For the performance of the method according to the present invention the samples to be analyzed are prepared for the binding inhibition test. For this the samples are diluted with an appropriate liquid if necessary. To analyze solid samples, the analytes are transferred into a liquid medium via convenient methods (e.g. extraction, dilution, milling, etc. . . . ). Afterwards, defined volumes of the liquid assays are pre-incubated with the appropriate ligands. Now and in the following, ligands are understood to be substances that have adequate structures of recognition for the sufficient specific detection and binding of analytes of the assay that are to be analyzed. For this, e.g. antibodies, aptameres, antigenes, coated beads, etc. can be used. According to the present invention, the ligands carry appropriate markers for the later detection that can be activated by means of an evanescent field. These can be, e.g. fluorescence dyes, quantum dots, etc. The pre-incubation of the sample is finished either after reaching the equilibration or after the run off of a previously defined period of time.

As soon as the pre-incubation is finished, the pre-treated sample is brought into contact with the coated support. For this the use of a flow method is particularly preferred. For this purpose the assay is led through a flow cell in or on which the coated layer is to be found, so that the assay is exposed to the coated surface. In a binding inhibition test, the ligands are able to bind in the assay by free binding sites to the corresponding molecular structures of the substances that are applied to the surface of the support. After the course of a certain time period, the incubation can be finished by flushing of the support.

When the incubation is finished, light is conducted from an adequate light source via total reflection through the support. It is particularly preferred to use light of a laser which is generated, e.g. from light emitting diodes (LED), laser diodes or lasers. In case of the total reflection, an evanescent field is generated on the phase boundary by which the markers settled on the surface of the support are activated. At this, the used marker and the used light have to be coordinated, so that an activation can occur. During this activation the marker emits its characteristic light that is detected by a detector. Especially photodiodes or CCD elements can be used as a detector. To avoid inaccurate test results by measuring of extrinsic light, adequate filters can be placed between the support and the detector. To lead, guide or modulate the light incident on the support and arriving on the detector, additional coupling and uncoupling elements, e.g. in the form of mirrors, lenses or optical fibers can be placed between the light source and the support and/or between the support and the detector.

The detected light intensities can now be analyzed and serve as a basis for the quantitative determination of the analyzed analytes. For this purpose, an evaluator, especially a computer can be used, so that the analysis is automated. According to the present invention, further procedural steps can be automatically controlled, too, like the preparation of the assay and the measuring step. In a particularly preferred embodiment the whole method is automatically controlled.

Different analytes, e.g. hormones, antibiotics, pesticides, pharmaceuticals, drugs and other molecules or molecular complexes, can be quantified according to the method of the present invention. In doing so, different liquids of different nature can be analyzed, e.g. drinking water, fruit juices, milk, serum, blood plasma, urine, etc. For every ascertained application, ligands are chosen in regard to the respective analytes with adequate structures of recognition, substances for the surface of the support with adequate molecular structures as well as an adequate preparation of the assay. The flexibility of the method allows its application in different fields: from food monitoring, to the is analysis of water, and to clinical diagnostics.

Compared to common methods, various analytes can be detected simultaneously faster, more sensitive and cheaper in different liquid mediums by the method according to the present invention.

More advantages, features and applications of the invention are described below based on the examples and referring to the drawings. The drawings show:

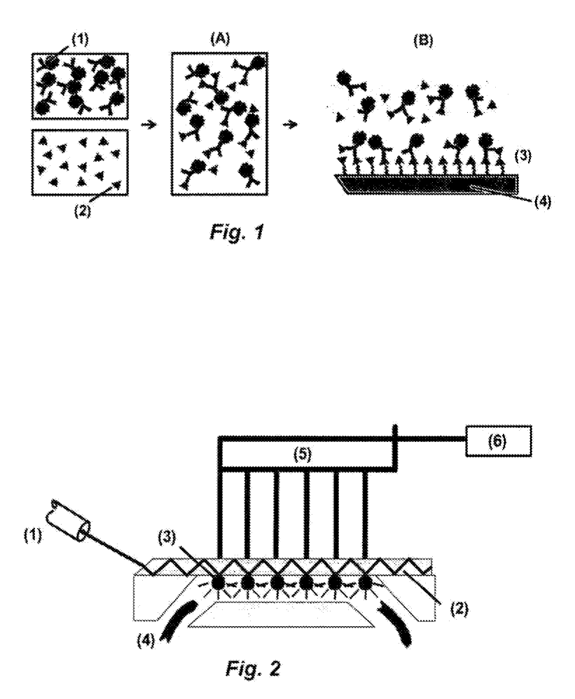

FIG. 1: The principle of the binding inhibition test as used in the method according to the present invention shown on the example of an immunoassay; thereby the antibodies 1 serve as ligands for the analyte 2; A: In the first step, the pre-incubation takes place in which to the assay containing analyte 2 the antibody 1 is added; B: In the second step, the assay is pumped over the support 4; the antibodies 1 can now bind with their free binding sites to the modified surface 3; after finishing the incubation, detection takes place.

FIG. 2: Schematic construction of a possible device for performing the method according to the present invention: the light is coupled from light source 1 into the glass medium 2 and via total reflection guided inside of the support 2; on the surface of the support which is coated with adequate substances 3 to specifically detect the analyte, evanescent field is build up nearby the surface; the assay can be contacted via a flow cell 4 to the surface of the support; after the incubation of the prepared assay, detection of the emitted light takes place via a detector 5; the latter passes the measuring data as detected to an analytical is workstation 6.

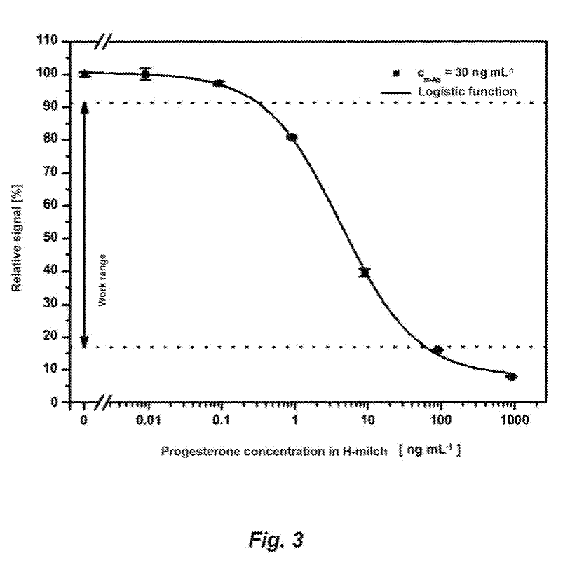

FIG. 3: calibration curve for progesterone in UT-IT-milk; concentrations of progesterone have been detected between 0.009 up to 900 ng ml⁻¹ (six assembly levels); the antibody was employed in each assay with a concentration of 30 ng ml⁻¹; a detection limit of 46 pg ml⁻¹ could be achieved.

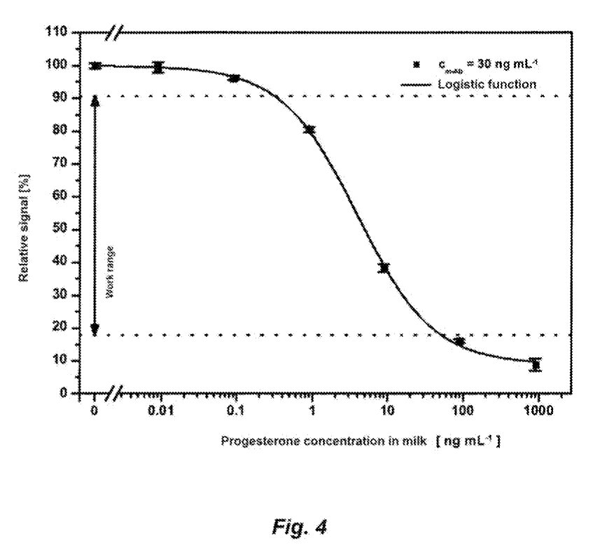

FIG. 4: calibration curve for progesterone in fresh milk; concentrations of progesterone have been detected between 0.009 up to 900 ng ml⁻¹ (six assembly levels); the antibody was employed in each assay with a concentration of 30 ng ml⁻¹; a detection limit of 56 pg ml⁻¹ could be achieved.

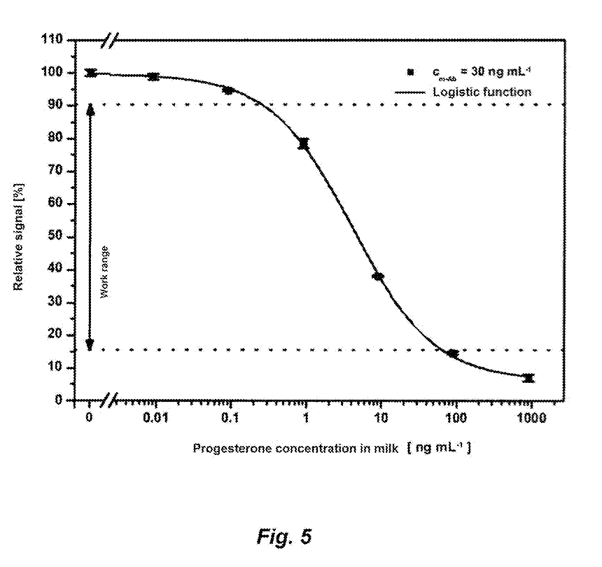

FIG. 5: calibration curve for progesterone in raw milk; concentrations of progesterone have been detected between 0.009 up to 900 ng ml⁻¹ (six assembly levels); the antibody was employed in each assay with a concentration of 30 ng ml⁻¹; a detection limit of 52 pg ml⁻¹ could be achieved.

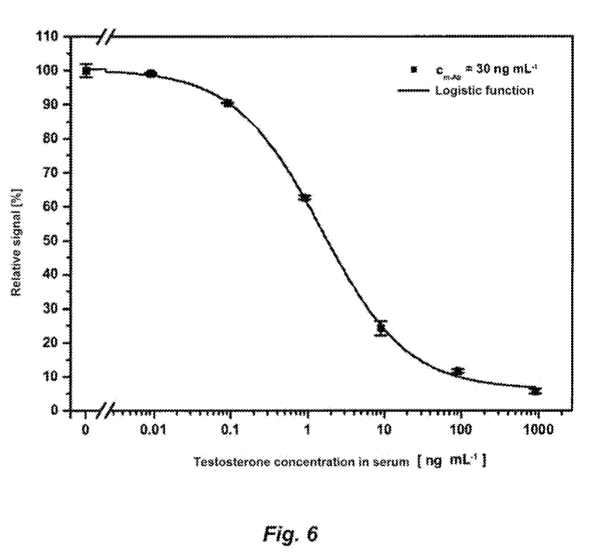

FIG. 6: calibration curve for testosterone in beef-serum; concentrations of testosterone have been detected between 0.009 up to 900 ng m11 (six assembly levels); the antibody was employed in each assay with a concentration of 30 ng ml⁻¹; a detection limit of 309 pg ml⁻¹ could be achieved.

### EMBODIMENTS

### Example 1

### Detection of Progesterone in Milk

In this embodiment the hormone progesterone is quantified in three different types of milk (UHT-milk, fresh milk and raw milk). At this, detection limits between 46 and 56 pg ml⁻¹ were achieved.

Consumption chemicals were procured by Sigma-Aldrich and Merck KGaA. The hormone was bought as VETRANAL® Standard at Riedl-de Haen Laboratory Chemicals GmbH & Co. KG. The monoclonal IgGl antibody, anti-progesterone, was acquired at Acris Antibodies GmbH. The used fluorescent marker CyDye™ Cy5.5 was purchased via Amersham Biosciences Europe GmbH. The aminodextran Amdex™ with a molecular weight of 40,000 Dalton was bought at Helix Research Company. The progesterone derivative for the immobilization on the surface of the support was synthesized.

The basic device consists of a 1 ml reciprocating piston injection device with a T-Valve for the Cavro-Modul XL 3000 of Tecan; a flushed loop consisting of a Teflon tube with ca. 2 ml total volume of Ismatec; a 6-Stroke-Valve with a flux-cell-tube (0.7 ml total volume) of Ismatec; a flow cell made of plexiglass with milled flow duct and Swagelok-circuit points for the intake and the outlet of proliquid; a bulcoptic supporting stand made of BK7 glass with a value of 60×14×1.5 mm of Desag, at which the bevel of 45° and the burnishing was carried out by PE Applied Biosystems; a modulated laser diode with a wavelength of 635 nm and 15 mW capacity of Coherent; six polymer fibers with a numeric aperture of 0.46; six edge filters 680 AELP with a diameter of 25 mm, a size of 4.5 mm and a maximal transmission of 90% of Omega Optical; six photodiodes with integrated pre-amplifier OSI 5-100M/1K and 50 M/2 K of Eurodis and electronics with lock-in-amplifier.

To prepare the assays, the auto sampler HTS PAL of CTC Analytics was used. It mainly consists of one movable 1 ml injecting device, a wash station, an inject-load-valve (Valco) with is six circuit points, a specimen holder for 98 1-ml-samples and one specimen holder for five 10-ml-samples. The software program Cycle Composer controls the auto sampler via a separate computer. The communication of both PC's is achieved by a relay-card. The auto sampler independently mixes the samples and injects them via the Valco-valve in the own flushed loop (Teflon tube with 960 ttl, Ismatec).

For the coating of the glass-support, two different methods are used: one for the single analyte measurements and the other for multi-analyte measurements. In the case of the single analyte measurements, the support is coated completely, whereas the multi-analyte measurements require a modification of the support in a spatially resolved manner by using micro dosing appliance. First of all the surface of the glass is purified and activated in both cases. Therefor the glass plates are put in a fresh Piranha-dilution for 30 min and next they are well washed with deionized water. After the drying in a nitrogen-flow, the support is lined with 50 ill GOPTS, a second one is put over it (sandwich technique) and both are mounted in a dry chamber. After 60 min, the supports are rapidly washed with dry acetone and dried under a nitrogen flow. For the complete coating, the activated supports are covered with 50 p.1 aminodextran-water-solution and folded up (sandwich technique). They are mounted in a water vapor atmosphere overnight. Afterwards they are washed with deionized water and dried. Now, the reaction with the derivative follows. For that purpose ca. 5 mg of the derivative is dissolved in little dry DMF and mixed with the 1.1-times the mole-amount DCC in DMF. This is solution is put on the support and again stored by means of the sandwich technique in a DMF-saturated chamber for at least five hours. Then the supports are first washed with DMF and then with deionized water.

For the modification performed in a spatially resolved manner, the GOPTS-activated support is dripped by means of the microdrop dosing system with a conjugate consisting of the derivative and aminodextran. The diameter of the spots amounts 3 mm and the distance of the spots 6.5 mm. The conjugate is produced from the active ester of the derivative and aminodextran (40 kD). For this purpose, ca. 5 mg of the derivative is dissolved in anhydrous DMF, and 1.1-times the mole-amount of NHS and 1.5-times the mole-amount DCC (each dissolved in DMF) are added. To form the conjugate, the aminodextran is dissolved in a mixture of a carbonate-buffer of pH 9.5 and DMF (1:1). 0.125 mole equivalent active ester are added to the AMD-solution. The solution is agitated overnight. Afterwards the conjugate is precipitated with methanol, washed and freeze-dried. The conjugate is dissolved in deionized and filtered water (2 mg ml⁻¹). The support is dripped with this solution by the micro dosing appliance.

The optical setup of the device consists of a laser diode which has a distance of about 2-5 cm from the support. The laser light is coupled into the glass-support via its beveled edge. The beam is further guided inside of the support by total reflection. The reflection points are located in distance of approximately 6.5 mm. At these sites, an evanescent field emerges near the surface in the flow cell. Here, fluorescent dye can be activated. On the back side of the support, polymer fibers are guiding the fluorescence over edge filters to the photodiodes. Because of the lock-in-technique as used the laser light is modulated, and only the arriving, appropriately modulated radiation is detected.

The sample is mixed by the HTS PAL auto sampler and injected into the flushed loop of the Valco-valve. Afterwards the infusing pump pumps the sample slowly over the flow cell. The antibody will be added directly into the sample before the measuring takes place. This has several advantages: first, the time interval between mixing and measuring remains always unchanged; second, the antibody is preserved because it stays relatively short in possibly aggressive matrices; and third, the antibody can be stored chilled in a reserve container without chilling the whole sample plate.

All concentrations of the analytes are measured at least threefold when calculating the analytic criteria of quality of calibrations. Therefore the calibration can be depicted more robust. The blank value is determined nine times. From the achieved data of the replica the blank value, the standard deviation and from those of the blank value measuring the detection limit are determined. The limit of quantification is calculated of the tenfold blank value standard deviation. The recovery rate for test assays adds up to the average value of the replica measurements and the true value.

The calibration in diluted milk (1:10) results from a 6-tier design with a progesterone concentration of 0.009 up to 900 nh ml⁻¹ in detection limits between 46 and 56 pg mL-I depending on the used type of milk (UHT-milk, fresh milk and raw milk). The achieved calibration curves are shown in FIG. 3 (UHT-milk), FIG. 4 (fresh milk) and FIG. 5 (raw milk).

Based on the calibration curves, real assays have been measured. Here, the following analyte concentrations were found at which it has to be considered that the calibration was done with fresh milk that already contained progesterone of about 1.0 ng m11.

| | | Concentration of |
|---|---|---|
| Sample | Rel. Signal % | Progesterone [ng ml⁻¹] |
| 1 | 79.74 | 10.17 |
| 2 | 74.10 | 14.91 |
| 3 | 84.10 | 7.15 |
| 4 | 92.25 | 2.73 |
| 5 | 68.29 | 21.04 |
| 6 | 59.73 | 33.32 |
| 7 | 42.43 | 81.97 |
| 8 | 80.08 | 9.91 |
| 9 | 84.36 | 6.98 |
| 10 | 83.79 | 7.34 |
| 11 | 75.18 | 13.92 |
| 12 | 92.22 | 2.75 |
| 13 | 83.03 | 7.84 |
| 14 | 84.17 | 7.11 |
| 15 | 95.00 | 1.57 |

### Example 2

### Detection of Testosterone in Beef-Serum

In this embodiment, the hormone testosterone is quantified in beef-serum.

Consumption chemicals were procured by Sigma-Aldrich and Merck KGaA. The hormone testosterone was bought as VETRANAL Standard at Riedl-de Haen Laboratory Chemicals GmbH & Co KG. The monoclonal IgGI antibody, anti-testosterone, was acquired at Acris Antibodies GmbH. The used fluorescent marker CyDye™ Cy5.5 was purchased via Amersham Biosciences Europe GmbH. The aminodextran Amdex™ with 40,000 Dalton molecular weight was bought at Helix Research Company. The testosterone derivative (Testosterone 3-(4-carboxymethypoxim) was procured via Sigma-Aldrich for the immobilization on the surface of the support.

The basic device and the optical setup of the device correspond to the system as used in Example 1. The auto sampler HTS PAL of CTC Analytics was used for the preparation of the samples corresponding to the method explained in Example 1. The preparation of the glass support is based on the method used for the complete coating of a single analyte measuring as described in Example I. The preparation of the sample and the method of measuring, as well as the calculation of the analytic characteristics, were made corresponding to Example 1.

The calibration in beef-serum resulted from a 6-tier design, at which the testosterone concentration of 0.009 up to 900 ng ml⁻¹ had been chosen. Here, a detection limit of 309 pg ml⁻¹ was achieved. The acquired calibration curve is shown in FIG. 6.

1-28. (canceled)

29. A method of the quantitative determination of analytes in liquid samples with the following steps:
a) incubation of the sample with adequate ligands provided with markers in the form of at least one antibody whereby the latter is directly added just before the determination starts, and the markers emit light as a result of an activation by an adequate evanescent field, and the ligands are able to sufficiently and specifically recognize and bind the analytes that should be quantified in the sample;
b) incubation of the sample as treated in the previous step with a light guiding support that is coated with adequate substances whereby these substances are able to specifically recognize and bind the ligands of the treated sample;
c) transfer of the light radiated from at least one light source through the support by total reflection whereby an evanescent field is build up on the boundary phases of the support;
d) detection of the light emitted by the markers as a result of an activation by an evanescent field by at least one detector;
e) quantitative determination of analytes in the sample based on the light intensities as measured by at least one detector; whereby at least one substance is applied on a support that is appropriate to the analytes or analyte-derivates to be quantified, and that at first the surface of the support is activated for the quantitative determination of the analyte(s), and afterwards is modified with 3-glycidyloxypropyltrimethoxysilane in such a manner that the surface of the support is lined with it and covered with a further support whereby an arrangement referring to a sandwich results, which is dried, and that subsequently the treatment of the supports is continued for the absorption of the analyte(s) or the analyte-derivative(s) after a predetermined time by washing with an appropriate liquid.

30. The method as defined in claim 1 wherein the liquid sample contains a physiological liquid.

31. The method as defined in claim 30 wherein the physiological liquid is selected from the group consisting of serum, blood plasma, urine, saliva, sperm or mixtures of thereof.

32. The method as defined in any one of the claims 1 to 3 for the quantitative single analyte determination whereby the activated and modified supports are completely coated by using aminodextran and allowed to repose as a sandwich before the reaction with the analyte or the analyte-derivative.

33. The method as defined in claim 32 wherein the reaction with the analytes or analyte-derivatives is performed by the sandwich-technique and that the sandwich is allowed to repose after the reaction.

34. The method as defined in claim 29 for the quantitative multianalyte determination whereby the activated and modified supports are dropped with a conjugate consisting of the analyte or analyte-derivate and aminodextran.

35. The method as defined in claim 34 wherein the diameters of the dropped areas have a size of about 3 mm and that the distance between those areas is about 6.5 mm.
